(19) United States
(12) Patent Application Publication
(10) Pub. No.: US 2021/0369728 A1
(43) Pub. Date: Dec. 2, 2021

(54) OPHTHALMIC COMPOSITION OF BRINZOLAMIDE AND BRIMONIDINE

(71) Applicant: MANKIND PHARMA LTD., New Delhi (IN)

(72) Inventors: Geoffrey Gnana Jeba JESUDIAN, Manesar (IN); Satish Chandra UPADHYAY, Manesar (IN); Amit BANSAL, Manesar (IN); Anil KUMAR, Manesar (IN)

(21) Appl. No.: 17/335,597

(22) Filed: Jun. 1, 2021

(30) Foreign Application Priority Data
Jun. 2, 2020 (IN) 202011023076

(51) Int. Cl. A61K 31/542; A61K 9/00; A61K 31/498

(52) U.S. Cl. CPC A61K 31/542; A61K 31/498; A61K 9/0048

(57) ABSTRACT

The present invention relates to an ophthalmic composition comprising brinzolamide or pharmaceutically acceptable salts thereof, and brimonidine or pharmaceutically acceptable salts thereof, and one or more pharmaceutically acceptable excipients. It further relates to a method of preparing such compositions and their use for the reduction of elevated intraocular pressure in patients with open angle glaucoma or ocular hypertension.

### FIELD OF THE INVENTION

The present application relates to an ophthalmic composition comprising brinzolamide or pharmaceutically acceptable salts thereof, and brimonidine or pharmaceutically acceptable salts thereof, and one or more pharmaceutically acceptable excipients. It further relates to a method of preparing such compositions and their use for the reduction of elevated intraocular pressure in patients with open angle glaucoma or ocular hypertension.

### BACKGROUND OF THE INVENTION

Brinzolamide is described chemically as (R)-(+)-4-Ethylamino-2-(3-methoxypropyl)-3, 4-dihydro-2H-thieno [3, 2-e]-1, 2-thiazine-6-sulfonamide-1, 1-dioxide.

Brimonidine tartrate is described chemically as 5-bromo-6-(2-imidazolidinylideneamino) quinoxaline L-tartrate.

The fixed combination of brinzolamide (1%) and brimonidine tartrate (0.2%) is currently marketed as ophthalmic suspension under the brand name Simbrinza®.

U.S. Pat. Nos. 6,316,441 and 6,242,442 disclose combinations of brinzolamide and brimonidine formulated as topical ophthalmic suspensions having brinzolamide in the formulations at concentrations of 1.0%-2.0% by weight, and brimonidine in the formulations at concentrations of 0.01%-0.2% by weight. These patents also disclose method for treating ocular conditions selected from the group consisting of glaucoma, occlusion conditions, diabetic retinopathy, and neovascularization which comprises administering a pharmaceutically effective amount of brinzolamide and brimonidine.

U.S. Pat. Nos. 9,044,484 and 9,421,265 disclose multi-dose ophthalmic compositions containing brinzolamide, brimonidine, two or more different polyols in conjunction with borate and a low concentration of benzalkonium chloride.

U.S. Publication No. 2014294750 A1 discloses aqueous pharmaceutical compositions containing two or more different polyols in conjunction with borate and a preservative selected from polymeric quaternary ammonium compound, hydrogen peroxide or a combination thereof.

U.S. Pat. No. 6,071,904 discloses ophthalmic suspensions containing brinzolamide, tyloxapol, carbomer 974P, edetate disodium, benzalkonium chloride, mannitol, sodium chloride and purified water.

PCT Publication No. WO 2019091596 A1 discloses ophthalmic composition comprising a combination of brinzolamide or pharmaceutically acceptable salt thereof and brimonidine or a pharmaceutically acceptable salt thereof together with a polyol-borate system comprising a single polyol, a borate and an antimicrobial preservative.

PCT Publication No. WO 2017099207 A1 discloses ophthalmic solution containing brimonidine and/or salt thereof and brinzolamide and/or salt thereof, wherein the product is contained in a transparent container having a maximum transmittance of light having a wavelength of 360 to 460 nm of 67% or less and a maximum transmittance of light having a wavelength of 600 to 680 nm of 78% or less.

PCT Publication No. WO 2017217450 A1 discloses ophthalmic solution containing brimonidine and/or salt thereof, brinzolamide and/or salt thereof, a carboxyvinyl polymer, and a dihydric alcohol and/or a trihydric alcohol, wherein the product is contained in a transparent container having a maximum transmittance for light having a wavelength of 360 to 460 nm of 67% or less and a maximum transmittance for light having a wavelength of 600 to 680 nm of 78% or less.

U.S. Pat. No. 10,517,869 discloses aqueous ophthalmic composition comprising brimonidine tartrate, hydroxypropyl methylcellulose and benzododecinium halide which is devoid of anionic cellulosic polymer.

U.S. Pat. No. 8,388,941 discloses multi-dose, self-preserved ophthalmic composition comprising a therapeutically effective amount of an ophthalmic therapeutic agent selected from the group consisting of beta-blockers, carbonic anhydrase inhibitors, prostaglandin analogs, neuroprotectants, anti-angiogenesis agents, quinolones, anti-inflammatory agents, growth factors, immunosuppressant agents and anti-allergic agents, borate/polyol complex and zinc ions in amounts sufficient to enhance the antimicrobial activity of the compositions, such that a conventional antimicrobial preservative is not required.

PCT Publication No. WO 2019235456 A1 discloses aqueous liquid preparation containing chlorobutanol and brimonidine and/or salt thereof that can suppress a decrease in pH over time and can have excellent formulation stability.

The main objective of the present application is to develop a stable ophthalmic formulation that is free of microbes during storage and for the duration of use; such formulation would provide a significant improvement over the prior art formulations.

The present invention provides an effective and safe topical ophthalmic composition containing a combination of drugs which has increased stability, fewer side effects as compared to other ophthalmic solutions available in the market.

### SUMMARY OF THE INVENTION

In main aspect, the present invention provides a thermodynamically stable ophthalmic composition comprising Brinzolamide or a pharmaceutically acceptable salt thereof, and Brimonidine or pharmaceutically acceptable salt thereof.

In another aspect, the present invention provides an ophthalmic pharmaceutical composition comprising brinzolamide or its pharmaceutically acceptable salts, and brimonidine or its pharmaceutically acceptable salts, with one or more pharmaceutically acceptable excipients, wherein the pharmaceutical composition is free of borate.

In another aspect, the present invention provides an ophthalmic pharmaceutical composition comprising brinzolamide or its pharmaceutically acceptable salts, brimonidine or its pharmaceutically acceptable salts, 0.01% w/v of benzalkonium chloride with one or more pharmaceutically acceptable excipients, wherein the pharmaceutical composition is free of borate.

In another aspect, the present invention provides an ophthalmic pharmaceutical composition comprising brinzolamide or its pharmaceutically acceptable salts, and brimonidine or its pharmaceutically acceptable salts, with one or more pharmaceutically acceptable excipients, wherein brimonidine or its pharmaceutically acceptable salts is in dissolved form and Brinzolamide or its pharmaceutically acceptable salts is suspended or dispersed in the composition, and wherein the pharmaceutical composition is free of borate.

In another aspect, the present invention provides a topical composition for ophthalmic application for use in lowering intraocular pressure in a patient comprising an ophthalmic composition comprising brinzolamide or its pharmaceutically acceptable salts, and brimonidine or its pharmaceutically acceptable salts, with one or more pharmaceutically acceptable excipients, wherein the composition is free of borate.

In another aspect, the present invention provides an aqueous sterile, ophthalmic pharmaceutical composition for lowering intraocular pressure in a patient suffering from elevated intraocular pressure comprising brinzolamide or its pharmaceutically acceptable salts thereof, brimonidine or its pharmaceutically acceptable salts thereof, a polymer, a buffer, a preservative along with one or more pharmaceutically acceptable excipient, wherein the pharmaceutical composition is in the form of suspension.

In another aspect, the present invention provides an ophthalmic pharmaceutical composition comprising brinzolamide and brimonidine tartrate along with one or more pharmaceutically acceptable excipients, wherein the one or more pharmaceutically acceptable excipients are selected from the group comprising buffer, polyol, tonicity adjusting agent, preservative, chelating agent, antioxidant, surfactant, co-solvent, viscosity modifying agent/suspending agent, vehicle, pH adjusting agent and/or combination thereof.

In another aspect, the present invention provides a method for the reduction of elevated intraocular pressure (IOP) in patients with open-angle glaucoma or ocular hypertension, wherein the method comprises administering a pharmaceutical composition comprising brinzolamide and brimonidine tartrate and one or more pharmaceutically acceptable excipients, wherein the pharmaceutical composition is free of borate.

In another aspect, the present invention provides a process for preparing an ophthalmic composition comprising brinzolamide and brimonidine tartrate and one or more pharmaceutically acceptable excipients.

### DESCRIPTION OF THE INVENTION

In main embodiment, the present invention provides a stable ophthalmic composition suitable for topical administration to the eye containing Brinzolamide or pharmaceutically acceptable salt thereof, and Brimonidine or a pharmaceutically acceptable salt thereof, along with one or more polyol, buffer and preservative agent.

Ophthalmic suspensions are sterile, free form particles and especially prepared for instillation into the eye. Considerations in preparing ophthalmic solutions involve clarity, tonicity, pH/buffer, sterility, preservatives, antioxidants, viscosity enhancers and proper packaging.

Applicant has tried various excipients and combination of excipients to develop an ophthalmic pharmaceutical composition of Brinzolamide and Brimonidine tartrate along with one or more pharmaceutically acceptable excipients, which is not only stable but also provide sufficient efficacy. While working on the present application, applicant has found that the stable pharmaceutical composition comprising Brinzolamide and Brimonidine tartrate can be achieved by using combination of one or more polyol, buffer and benzalkonium chloride in a specific ratio.

Accordingly, applicant has developed an ophthalmic pharmaceutical composition comprising brinzolamide or its pharmaceutically acceptable salts, and brimonidine or its pharmaceutically acceptable salts, with one or more pharmaceutically acceptable excipients, wherein the pharmaceutical composition is free of borate.

As used herein, the term “treat” is to relieve, reduce or alleviate at least one symptom of a disease in a subject. For example, the term “treat” may mean to reduce or alleviate elevated intraocular pressure and/or to reduce or prevent further damage or loss of retinal ganglion cells. For example, treatment can be diminishment of one or several symptoms of a disorder or complete eradication of a disorder.

As used herein, the term “topical application” means application by way of a liquid, solution, suspension, gel, cream or ointment to the external corneal surface of a subject.

As used herein, the term “borate” shall refer to boric acid, salts of boric acid, borate derivatives and other pharmaceutically acceptable borates, or combinations thereof. Preferably the borates are selected from boric acid, sodium borate, potassium borate, calcium borate, magnesium borate, manganese borate, and other such borate salts.

As used herein, the term “phosphate buffer” shall refer to phosphoric acid, salts of phosphoric acid, phosphoric acid derivatives and other pharmaceutically acceptable phosphates, or combinations thereof. Phosphate buffer includes, but not limited to, phosphoric acid, monobasic sodium phosphate, dibasic sodium phosphate, potassium phosphate and other phosphate salts.

As used herein, the term “polyol” includes sugars, sugar alcohols, sugar acids and uronic acids. Preferred polyols are sugars, sugar alcohols and sugar acids, including, but not limited to mannitol, glycerin, glycerol, maltitol, lactitol, isomalt, erythritol, xylitol, sorbitol, polyethylene glycol, propylene glycol.

In an embodiment of the present invention, there is provided an ophthalmic composition comprising brinzolamide or pharmaceutically acceptable salts thereof, wherein the composition comprises 0.5% to 2.0% w/v of brinzolamide, more preferably 1.0% w/v of brinzolamide or pharmaceutically acceptable salts thereof.

In another embodiment of the present invention, there is provided an ophthalmic composition comprising brimonidine or pharmaceutically acceptable salts thereof, wherein the composition comprises 0.01% to 0.4% w/v of brimonidine, more preferably 0.2% w/v of brimonidine or pharmaceutically acceptable salts thereof.

In another embodiment of the present invention, there is provided an ophthalmic composition comprising brinzolamide and brimonidine, wherein the brinzolamide and brimonidine are present in any known polymorphic form(s).

In another embodiment of the present invention, there is provided a pharmaceutical composition, wherein the composition has a pH in the range of about 5.0 to 8.0.

In another embodiment of the present invention, there is provided a pharmaceutical composition, wherein the composition is in the form of a suspension, solution, emulsion, gel, dispersion or nanodispersion.

In yet another embodiment, the present application provides a pharmaceutical composition, wherein the composition is free of benzalkonium chloride.

In another embodiment, the present invention provides a pharmaceutical composition, wherein the composition is free of borate.

In another embodiment, the present invention provides an ophthalmic pharmaceutical composition comprising brinzolamide or its pharmaceutically acceptable salts, brimonidine or its pharmaceutically acceptable salts, with one or more pharmaceutically acceptable excipients, wherein the pharmaceutical composition further comprises benzalkonium chloride in an amount from 0.0036% to 0.02% w/v of the composition, more preferably 0.01% w/v of the composition, and wherein the pharmaceutical composition is free of borate.

In another embodiment, the present invention provides a pharmaceutical composition, wherein the composition is filled into a single-dose container or multi-dose container and wherein the suitable containers include, but not limited to, bottles, vials or ampoules.

In another embodiment, the present invention provides a pharmaceutical composition, wherein the composition is filled in three piece bottle of suitable capacity plugged with nozzle and seal with cap. Pharmaceutically acceptable packaging materials include, but are not limited to, low density polyethylene (“LDPE”), high density polyethylene (“HDPE”), polypropylene, polystyrene, polycarbonate, polyesters (such as polyethylene terephthalate and polyethylene naphthalate), nylon, polyvinyl chloride, poly(vinylidine chloride), poly(tetrafluoroethylene) and other materials known in the literature.

Typically, the bottle may be made from Low Density Polyethylene (LDPE), Linear Low Density Polyethylene (LLDPE), High Density Polyethylene (HDPE), Polypropylene (PP) and the like or a combination thereof. Typically, the nozzle/cap may be made of low density polyethylene (LDPE), linear low density polyethylene (LLDPE), high density polyethylene (HDPE), polypropylene (PP) and the like or a combination thereof. Alternatively, the nozzle may be made of a hydrophobic material or may be coated with a hydrophobic material such as a fluoropolymer like Teflon (polytetrafluoroethylene) and the like.

The pharmaceutical compositions or the active ingredient as per the present invention can be sterilized using any of the known methods of sterilization, such as filtration, moist heat, dry heat, gas sterilization or irradiation (gamma and electron beam) or combination thereof. The container in which composition is filled can be sterilized using gamma irradiation or ethylene oxide or pre-acetic acid or any other conventional method of sterilization. The compositions can be terminally sterilized also.

In another embodiment, the present invention provides a pharmaceutical composition, wherein the composition has total impurities less than the maximum allowed limit as per ICH guidelines.

In another embodiment, the present invention provides a pharmaceutical composition, wherein the one or more pharmaceutically acceptable excipients are selected from the group comprising buffer, polyol, tonicity adjusting agent, preservative, chelating agent, antioxidants, surfactant, co-solvent, viscosity modifying agent/suspending agent, vehicle, pH adjusting agent and/or combination thereof.

In another embodiment, the suitable preservatives used in the composition include cetrimide, polyquaternium-1, thiomersal or thimerosal, acids and their pharmaceutically acceptable salts such as sorbic acid, potassium sorbate, boric acid, borax, sodium perborate NaBO3, salicylic acid, benzoic acid, lactic acid, acetic acid; S.O.C (stabilized oxychloro complex)/purite, S.C.P (stabilized chlorite peroxide), phenylethanol, benzalkonium chloride, benzododecinium bromide (BDD), benzethonium chloride, cetrimonium chloride, methyl parahydroxybenzoate, polyquaternium ammonium chloride, polyaminopropyl, and hydrogen peroxide, parabens such as methyl paraben, propyl paraben, ethyl paraben, butyl paraben, perborates, phenol and its derivatives such as m-cresol and chlorocresol, benzyl alcohol, halogenated alcohols such as chlorobutanol and/or combinations thereof. The preservative is present in amount from 0.0036% to 0.05% w/v of the composition, more preferably 0.01% w/v of composition.

In another embodiment, the suitable viscosity modifying agent/suspending agents used in the composition include hydroxypropyl methylcellulose (HPMC), hydroxypropylcellulose (HPC), carboxymethylcellulose (CMC), methylcellulose (MC), hydroxyethylcellulose (HEC), cellulose and derivatives thereof, polycarbophil, polyoxyethylene glycol (PEG), hyaluronic acid (HA), amylase and derivatives thereof, amylopectins and derivatives thereof, dextran and derivatives thereof, polyvinylpyrrolidone (PVP), polyvinyl alcohol (PVA), and acrylic polymers such as derivatives of polyacrylic or polymethacrylic acid including hydroxylmethyl methacrylate (HEMA), carbomer such as carbomer 974 P or a mixture thereof. The viscosity modifying agent may be used in concentrations ranging from about 0.001% to about 5% w/v of the composition, preferably about 0.05% to about 4% w/v of the composition, more preferably about 0.2% to about 0.6% w/v of the composition.

In another embodiment, the present invention provides a pharmaceutical composition, wherein the buffers may be used in concentrations ranging from about 0.001% to about 4% w/v of the composition, more preferably about 0.01% to about 2% w/v of the composition. Suitable buffering agents include, but are not limited to, phosphates, such as sodium phosphate monobasic, sodium phosphate dibasic, phosphoric acid, citric acid, citrates such as sodium citrate, borate, acetic acid, acetates such as sodium acetate, lactic acid, sodium lactate, tartaric acid, sodium tartrate, tartrates, succinates, amino acids such as L-arginine, tromethamine and/or combinations/mixtures thereof.

In preferred embodiment, the present invention provides a pharmaceutical composition, wherein the composition comprises buffering agent in an amount of 0.001% to about 4% w/v of the overall composition, and most preferably the composition comprises borate as a buffering agent, and in an amount of 0.5% to about 1.0% w/v.

In another embodiment, the suitable osmotic/tonicity adjusting agents include propylene glycol, glycerol/glycerine, sodium chloride, potassium chloride, sodium bromide, calcium chloride, mannitol, sorbitol, dextrose, sucrose, mannose and the like and mixtures thereof. The osmotic agent is used in an amount to maintain the solution's osmolality compatible with respect to eye fluid. The tonicity adjusting agent may be used in concentrations ranging from about 0.01% to about 1% w/v of the composition.

In another embodiment, the present invention provides a pharmaceutical composition, wherein the composition comprises one or more polyol. Suitable polyols are sugars, sugar alcohols and sugar acids, including, but not limited to mannitol, glycerin, Maltitol, Lactitol, Isomalt, Erythritol, xylitol, sorbitol, polyethylene glycol, propylene glycol. The polyol may be used in concentrations ranging from about 0.01% to about 3% w/v of the composition.

In another embodiment, the present invention provides a pharmaceutical composition, wherein the composition is free of polyol.

In another embodiment, the present invention provides a pharmaceutical composition, wherein the composition is free of polyol and benzalkonium chloride.

In another embodiment, the present invention provides a pharmaceutical composition, wherein the composition is free of polyol, benzalkonium chloride and borate.

In another embodiment, the present invention provides a pharmaceutical composition, wherein brimonidine or its pharmaceutically acceptable salts is in dissolved form and Brinzolamide or its pharmaceutically acceptable salts is suspended or dispersed in the composition.

In another embodiment, the present invention provides a pharmaceutical composition, wherein the particle size of Brinzolamide in the composition is d₉₀ less than 20 μm.

In another embodiment, the present invention provides a pharmaceutical composition, wherein the viscosity of the suspension is less than 1000 cps, more preferably less than 500 cps and even more preferably less than 150 cps. Viscosity of the brinzolamide suspension is in the range of about 30 cps to about 120 cps.

In another embodiment, the suitable pH adjusting agents include acids and bases. Suitable acids to adjust the pH of the composition include, but are not limited to, hydrochloric acid, glacial acetic acid, citric acid, sulfuric acid, nitric acid and/or combinations thereof.

In another embodiment, the suitable bases to adjust the pH of the composition include, but are not limited to, sodium hydroxide, potassium hydroxide, barium hydroxide, sodium acetate, sodium phosphate, amino acid and/or combinations thereof.

In another embodiment, the suitable vehicles/diluents include water for injection, purified water, Ringer's solution, normal saline solution.

In another embodiment, the suitable antioxidants include, but are not limited to, sodium thiosulfate, sodium bisulfite, acetone sodium bisulfite, gentisic acid, gentisic acid ethanolamide, sodium formaldehyde sulfoxylate, thiourea, butylated hydroxyanisole, butylated hydroxy toluene, esters of gallic acid, ascorbic acid, salts of ascorbic acids such as ascorbyl palmitate, sodium ascorbate, retinoids and derivatives of vitamin A, acetylcysteine, thioglycerol, Vitamin E and its derivatives e.g. Vitamin E TPGS and/or combinations thereof. Antioxidant may be used in concentrations ranging from about 0.001% to about 5% w/v of the composition.

In another embodiment, the suitable surfactants include but are not limited to, hydrophilic surfactants, lipophilic surfactants, and mixtures thereof. The formulations can contain surface-active agents conventionally employed in topical formulations, such as oleic acid, lecithin, sorbitan trioleate, benzododecinium bromide, cetylpyridinium chloride, tyloxapol, polyoxyethylene sorbitan monolaurate, polyoxyethylene sorbitan monostearate, polyoxyethylene sorbitan mono-oleate, polyoxypropylene/polyoxyethylene block copolymers, polyoxypropylene/polyoxyethylene/ethylene diamine block copolymers, ethoxylated castor oil and/or combinations thereof. The surfactant may be used in concentrations ranging from about 0.01% to about 1% w/v of the composition.

In another embodiment, the suitable co-solvents include propylene glycol, polyethylene glycol, glycerine, glycerol and the like or mixtures thereof.

In another embodiment, the suitable chelating agents include edetate disodium, ethylenediamine tetracetic acid, edetic acid, disodium edetate dihydrate, diethylenetriamine pentaacetic acid and/or combinations thereof. The chelating agent may be used in concentrations ranging from about 0% to about 5% w/v of the composition.

In another embodiment, the present invention provides an ophthalmic pharmaceutical composition comprising brinzolamide or its pharmaceutically acceptable salts, brimonidine or its pharmaceutically acceptable salts, with one or more pharmaceutically acceptable excipients, wherein the pharmaceutical composition further comprises benzalkonium chloride in an amount of 0.0036% to 0.02% w/v of the composition, and borate in an amount of 0.55% to 1.0% w/v of the composition.

In another embodiment, the present invention provides a method for the reduction of elevated intraocular pressure (IOP) in patients with open-angle glaucoma or ocular hypertension, wherein the method comprises administering a pharmaceutical composition comprising brinzolamide and brimonidine tartrate and one or more pharmaceutically acceptable excipients.

In another embodiment, the present invention provides a process for preparing an ophthalmic composition comprising brinzolamide and brimonidine tartrate and one or more pharmaceutically acceptable excipients, wherein said process involves adding of both drugs and other excipients to suitable vehicle to form final composition followed by filling in a suitable container.

In another embodiment, the present invention provides process for the preparation of composition comprising brinzolamide or its pharmaceutically acceptable salt, and brimonidine or its pharmaceutically acceptable salt, and one or more pharmaceutically acceptable excipients, wherein said process comprises the steps of:

a) autoclaving the slurry of sterilized brinzolamide or its pharmaceutically acceptable salt containing milling beads and suitable surfactant;
b) milling the slurry of step a) using suitable milling technique to get a milled slurry;
c) preparing a solution of brimonidine or its pharmaceutically acceptable salt in suitable vehicle;
d) preparing a polymer slurry by adding the solution of step c) to a sterilized polymer phase;
e) aseptically mixing the milled slurry of step b) to polymer slurry of step d) and adjusting pH;
f) making up the volume using water of injection; and
g) aseptically filling the container.

In another embodiment, the suitable milling technique includes, but not limited to, ball mill, an attritor mill, a vibratory mill, and media mills such as a sand mill and a bead mill.

In another embodiment, drugs can be sterilized separately either alone or with other excipients optionally followed by milling and then adding it to the remaining excipients to form final composition.

The following examples will illustrate in more detail the various embodiments of the present application.

TABLE 1
Pharmaceutical composition of Brinzolamide and Brimonidine tartrate

| | Quantity % (w/v) (Formulations) | | | | |
|---|---|---|---|---|---|
| | Example | Example | Example | Example | Example |
| Ingredients | 1 | 2 | 3 | 4 | 5 |
| Brinzolamide | 1.0 | 1.0 | 1.0 | 1.0 | 1.0 |
| Brimonidine | 0.2 | 0.2 | 0.2 | 0.2 | 0.2 |
| tartrate | | | | | |
| Benzethonium | 0.0036-0.05 | — | — | — | — |
| chloride | | | | | |
| Benzododecinium | — | 0.0036-0.05 | — | — | — |
| bromide | | | | | |
| Benzalkonium | — | — | 0.0036-0.05 | 0.0036-0.05 | 0.0036-0.05 |
| chloride | | | | | |
| Propylene glycol | 0.01-3 | 0.01-3 | 0.01-3 | 0.01-3 | — |
| Carbomer 974P | 0.05-4 | 0.05-4 | 0.05-4 | 0.05-4 | 0.05-4 |
| Boric acid | 0.01-1 | 0.01-1 | — | — | 0.01-1 |
| Phosphate Buffer | — | — | 0.01-2 | — | — |
| Trisodium citrate | — | — | 0.01-0.5 | — | — |
| dihydrate | | | | | |
| Tromethamine | — | — | — | 0.2-2 | — |
| Mannitol | 0.01-2 | 0.01-2 | 0.01-2 | 0.01-2 | 0.01-2 |
| Sodium chloride | 0.1-1 | 0.1-1 | 0.1-1 | 0.1-1 | 0.1-1 |
| Tyloxapol | 0.01-0.1 | 0.01-0.1 | 0.01-0.1 | 0.01-0.1 | 0.01-0.1 |
| Hydrochloric acid | QS pH | QS pH | QS pH | QS pH | QS pH |
| | 5-8 | 5-8 | 5-8 | 5-8 | 5-8 |
| Sodium Hydroxide | QS pH | QS pH | QS pH | QS pH | QS pH |
| | 5-8 | 5-8 | 5-8 | 5-8 | 5-8 |
| Purified water | QS | QS | QS | QS | QS |

TABLE 2
Pharmaceutical composition of Brinzolamide and Brimonidine tartrate

| | Quantity % (w/v) (Formulations) | | | | |
|---|---|---|---|---|---|
| | Example | Example | Example | Example | Example |
| Ingredients | 6 | 7 | 8 | 9 | 10 |
| Brinzolamide | 1.0 | 1.0 | 1.0 | 1.0 | 1.0 |
| Brimonidine | 0.2 | 0.2 | 0.2 | 0.2 | 0.2 |
| tartrate | | | | | |
| Benzalkonium | 0.0036-0.05 | — | — | — | — |
| chloride | | | | | |
| Phenyl ethanol | — | 0.05-1 | — | 0.05-1 | — |
| Propylene glycol | 0.01-3 | — | — | — | — |
| Carbomer 974P | 0.05-4 | 0.05-4 | 0.05-4 | 0.05-4 | 0.05-4 |
| Boric acid | 0.01-1 | 0.01-1 | 0.01-1 | — | — |
| Tromethamine | — | — | — | 0.2-2 | 0.2-2 |
| Mannitol | — | — | — | — | — |
| Sodium chloride | 0.1-1 | 0.1-1 | 0.1-1 | 0.1-1 | 0.1-1 |
| Tyloxapol | 0.01-0.1 | 0.01-0.1 | 0.01-0.1 | 0.01-0.1 | 0.01-0.1 |
| Chlorobutanol | — | — | 0.5-5 | — | 0.5-5 |
| Hemihydrate | | | | | |
| Hydrochloric acid | QS pH | QS pH | QS pH | QS pH | QS pH |
| | 5-8 | 5-8 | 5-8 | 5-8 | 5-8 |
| Sodium Hydroxide | QS pH | QS pH | QS pH | QS pH | QS pH |
| | 5-8 | 5-8 | 5-8 | 5-8 | 5-8 |
| Purified water | QS | QS | QS | QS | QS |

TABLE 3
Pharmaceutical composition of Brinzolamide and Brimonidine tartrate

| | Quantity % (w/v) | | | | |
|---|---|---|---|---|---|
| | Example | Example | Example | Example | Example |
| Ingredients | 11 | 12 | 13 | 14 | 15 |
| Brinzolamide | 1.0 | 1.0 | 1.0 | 1.0 | 1.0 |
| Brimonidine | 0.20 | 0.20 | 0.20 | 0.20 | 0.20 |
| Tartrate | | | | | |
| Carbopol 974P | 0.40 | 0.40 | 0.40 | 0.41 | 0.45 |
| Tyloxapol | 0.025 | 0.025 | 0.025 | 0.025 | 0.025 |
| Tromethamine | — | 0.01-2 | — | — | 0.30 |
| Mannitol | 0.30 | 0.30 | 0.30 | 0.30 | 0.30 |
| Boric Acid | — | — | 0.30 | — | — |
| Propylene Glycol | 0.75 | 0.75 | 0.75 | 0.75 | 0.75 |
| Monobasic sodium | 0.40 | — | — | 0.40 | — |
| phosphate | | | | | |
| monohydrate | | | | | |
| Dibasic sodium | 0.16 | — | — | 0.16 | — |
| phosphate | | | | | |
| heptahydrate | | | | | |
| Sodium Chloride | 0.23 | 0.23 | 0.23 | 0.23 | 0.23 |
| Benzalkonium | 0.01 | 0.01 | 0.003 | 0.01 | 0.01 |
| Chloride | | | | | |
| Sodium Hydroxide | QS pH | QS pH | QS pH | QS pH | QS pH |
| | 5-8 | 5-8 | 5-8 | 5-8 | 5-8 |
| Hydrochloric Acid | QS pH | QS pH | QS pH | QS pH | QS pH |
| | 5-8 | 5-8 | 5-8 | 5-8 | 5-8 |
| Purified Water | QS | QS | QS | QS | QS |

The pharmaceutical composition of present invention was prepared by the process comprises five main steps, which are as follows:
1. Preparation of brimonidine tartrate containing vehicle
2. Preparation of Polymer Phase
3. Preparation of Tyloxapol solution
4. Preparation of API Slurry and size reduction
5. Bulk Preparation

### 1. Preparation of Brimonidine Tartrate Containing Vehicle:

a. Take 30% (of actual batch size) of water in a clean glass beaker.
b. Add slowly dispensed quantity of mannitol, propylene glycol, sodium chloride, benzalkonium chloride and phosphate buffer/tromethamine. Stir till clear solution is obtained before addition of next ingredient.
c. Add slowly dispensed quantity of non-sterile API (brimonidine tartrate) to it under continuous stirring.
d. Adjust the pH of solution to 5.0 to 8.0 with sodium hydroxide solution/Hydrochloric acid solution.
e. Make up the volume up to 40% of batch size.
f. Filter through 0.2-micron PES/PVDF filter.

### 2. Preparation of Polymer Phase:

a. Take 30% (of actual batch size) of water in a clean glass beaker.
b. Slowly add dispensed quantity of carbomer into the water.
c. Check the initial pH of the solution, and adjust the pH to 6.0-7.5 with sodium hydroxide solution/Hydrochloric acid solution.
d. Make up the volume with water up to 40% (of actual batch size).
e. Autoclave/In-situ sterilize the polymer phase at 121° C. for 15-30 min at 15 psi.

### 3. Preparation of Tyloxapol Solution:

a. Take sufficient quantity of water in a clean glass beaker.
b. Add slowly dispensed quantity of tyloxapol. Stir till clear solution is obtained.
c. Make up the volume up to required batch size.
d. Filter through 0.2-micron PES/PVDF filter.

### 4. Preparation of API Slurry and Size Reduction:

a. Take required quantity of tyloxapol solution in a clean glass beaker.
b. Add slowly dispensed quantity of sterile API (Brinzolamide) to it under continuous stirring.
c. Reduce the particle size of the API using bead mill using Polystyrene/Zirconium beads for specified duration.

### 5. Bulk Preparation:

a. Add the Brimonidine tartrate containing vehicle (40% of batch size) to previously sterilized polymer phase which is approximately 40% of batch size by weight.
b. Add the API slurry obtained in step D to the solution obtained in step 1.
c. Check the initial pH of the bulk and adjust the pH to 6.0-7.5 with sterile sodium hydroxide solution/Hydrochloric acid solution.
d. Make up the volume with water for injection up to 100.0% (of actual batch size)
e. Stir the solution for 2 hours in aseptic conditions.
f. Fill the final suspension aseptically in previously sterilized bottles, suitable for ophthalmic use.

The pharmaceutical composition of present invention was also prepared by the process comprises 7 main steps, which are as follows:
1. Preparation of sterile Tyloxapol solution
2. Preparation of carbopol slurry
3. Preparation and filtration of inactive phase
4. Mixing and sterilization of Carbopol slurry
5. Preparation of 1N NaOH/0.1N HCl solution
6. Neutralization of sterilized Carbopol slurry
7. Homogenization of drug suspension

### 1. Preparation of Sterile Tyloxapol Solution:

a. Take sufficient quantity of water in a clean glass beaker.
b. Add slowly dispensed quantity of tyloxapol. Stir till clear solution is obtained.
c. Make up the volume up to required batch size.
d. Filter through 0.2-micron PES/PVDF filter.

### 2. Preparation of Carbopol Slurry:

a. Take sufficient quantity of water in a clean glass beaker and add sodium chloride to make solution.
b. Slowly add dispensed quantity of carbomer into the solution with continuous mixing about 2 hours to form homogenous slurry.

### 3. Preparation and Filtration of Inactive Phase:

a. Take sufficient quantity of water in a clean glass beaker.
b. Add EDTA in water and stir till clear solution is obtained.
c. Add mannitol and benzalkonium chloride in solution and stir till clear solution obtained.
d. Filter the solution through 0.2-micron PES/PVDF filter

### 4. Mixing and Sterilization of Carbopol Slurry:

a. Take Carbopol slurry of step 2 and solution obtained in step 3 and makeup the suitable volume.
b. Mix for 10-20 min at 400±20 rpm.
c. Autoclave/In-situ sterilize at 121° C. for 15-30 min at 15 psi.
   5. Preparation of 1N NaOH/0.1N HCl solution:
a. Prepare 1N NaOH or 0.1N HCl solution.
b. Filter through 0.2-micron PES/PVDF filter to obtained sterile solution.

### 6. Neutralization of Sterilized Carbopol Slurry:

a. Add suitable amount of 1N NaOH solution to carbopol slurry obtained in step 4.
b. Mix using anchor type (40-50 rpm) or bottom mounted propeller (1300-1400 rpm) for 60±15 min.
c. Adjust the pH of solution at 7.5±0.4 from 1N NaOH or 0.1N HCl solution.
   7. Homogenization of drug suspension:
a. Take sterile Tyloxapol solution of step 1 and add dry heat sterilized API.
b. Mix for 10±2 min at 750±50 rpm and homogenize using suitable homogenizer.
c. Add Carbopol slurry under aseptic condition and mix for 20 min at 40 rpm using anchor type propeller.
d. Make up the volume and mix for 30-35 min at 43-45 rpm using anchor type propeller under aseptic condition.
e. Fill the final suspension aseptically in previously sterilized bottles, suitable for ophthalmic use.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. An ophthalmic pharmaceutical composition comprising brinzolamide or its pharmaceutically acceptable salts, and brimonidine or its pharmaceutically acceptable salts, with one or more pharmaceutically acceptable excipients, wherein the pharmaceutical composition is free of borate.

2. The ophthalmic pharmaceutical composition as claimed in claim 1, wherein the pharmaceutical composition comprises one or more preservative.

3. The ophthalmic pharmaceutical composition as claimed in claim 1, wherein the pharmaceutical composition comprises one or more preservative in an amount of 0.0036% to 0.05% w/v of the composition.

4. The ophthalmic pharmaceutical composition as claimed in claim 1, wherein the pharmaceutical composition comprises benzalkonium chloride in an amount of 0.01% w/v of the composition.

5. The ophthalmic pharmaceutical composition as claimed in claim 1, wherein the pharmaceutical composition has a pH in the range of about 5.0 to 8.0.

6. The ophthalmic pharmaceutical composition as claimed in claim 1, wherein the composition comprises one or more polyol.

7. An ophthalmic pharmaceutical composition comprising Brinzolamide or its pharmaceutically acceptable salts, Brimonidine or its pharmaceutically acceptable salts, with one or more pharmaceutically acceptable excipients, wherein the composition further comprises benzalkonium chloride in an amount of 0.0036% to 0.02% w/v of the composition, and borate in an amount of 0.55% to 1.0% w/v of the composition.

8. The ophthalmic pharmaceutical composition as claimed in claim 1, wherein Brimonidine or its pharmaceutically acceptable salts is in dissolved form and Brinzolamide or its pharmaceutically acceptable salts is suspended or dispersed in the composition.

9. The ophthalmic pharmaceutical composition as claimed in claim 1, wherein the composition is in the form of a suspension, solution, emulsion, gel, dispersion or nanodispersion.

10. An aqueous sterile ophthalmic pharmaceutical composition for lowering intraocular pressure in a patient suffering from elevated intraocular pressure comprising brinzolamide or its pharmaceutically acceptable salts, and brimonidine or its pharmaceutically acceptable salts, a polymer, a buffer, a preservative along with one or more pharmaceutically acceptable excipient, wherein the composition is free of borate.
